(19) United States
(12) Patent Application Publication
(10) Pub. No.: US 2023/0240815 A1
(43) Pub. Date: Aug. 3, 2023

(54) TWISTING INSTRUMENT

(71) Applicant: Bruno SPINDLER, Oppenau (DE)

(72) Inventors: Bruno SPINDLER, Oppenau (DE); Timo REUTER, Bruchsal (DE)

(73) Assignee: Bruno SPINDLER, Oppenau (DE)

(21) Appl. No.: 18/009,464

(22) PCT Filed: Jun. 3, 2021
(86) PCT No.: PCT/DE2021/000105

§ 371 Date: Dec. 9, 2022

(30) Foreign Application Priority Data
Jun. 11, 2020 (DE) DE1020200003492.6

(51) Int. Cl. A61C 8/00; B25B 15/00; B25B 23/14

(52) U.S. Cl. CPC A61C 8/0089; B25B 15/005; B25B 23/1415

(57) ABSTRACT

A twisting instrument for a screw-in component with a tool recess comprises a drive shaft, a neck, and a working part. The working part has—as a spherical head having a plurality of teeth—a spherical enveloping surface that is contacted at least both by the free end and by sections of the tooth heads of the teeth of the working part. The neck or the drive shaft has a predetermined breaking zone. The working part engages in the tool recess of the screw-in part in an overload-proof and reliable manner.

### TECHNICAL FIELD

The disclosure relates to a twisting instrument for a screw-in component with a tool recess, comprising a drive shaft, a neck and a working part.

### BACKGROUND

In dental implantology, among other things, an enossal implant body is often used to support the individual denture as part of the fabrication of a single-tooth denture. In this case, the implant body, a type of screw dowel, is screwed into an artificially created bore in the patient's jaw. The screwed-in implant body accommodates an implant post in the finished prosthesis. The latter is fastened against rotation in the implant body with a special clamping device, for example a screw or a special threaded bolt. A twisting instrument is used to fasten or remove the clamping device.

A superstructure forming the visible tooth crown is placed directly or indirectly on the implant abutment fastened in this manner, for example by bonding.

A screwdriving tool with which screws are screwed in for fastening abutments to an implant body is known from WO 2017/070 335 A1. The screwing tool has a driving part, a shaft and a working part. The working part has a large number of rounded teeth between which there are concave surfaces.

### SUMMARY

The present disclosure is based on the problem of improving a twisting instrument for a screw-in component in such a manner that the working part of the twisting instrument engages in the tool recess of the screw-in component in an overload-proof and reliable manner, wherein the center lines of the twisting instrument and the screw-in component enclose a deflection angle of zero or more angular degrees.

This problem is solved with the features of patent claim 1. In this case, the working part—as a spherical head having a plurality of teeth—has a spherical enveloping surface that is contacted at least both by the free end and by sections of the tooth heads of the teeth of the working part. The neck or the drive shaft has a predetermined breaking zone.

The disclosure provides a twisting instrument that can be used to screw together individual parts of a denture during the assembly and maintenance of a prosthetic denture. The screws, threaded bolts or the like required for the screw connection have a tool recess that forms a mechanical interface with the working part in the form of an angularly movable coupling. The ball-head-shaped, toothed working part, which has, for example, three to eight teeth, is integrally connected to a torsionally rigid drive shaft via one at least torsionally flexible neck. To use the twisting instrument, an actuating element that is usually manual is attached to the drive shaft. In the mechanical interface located between the drive shaft and the actuating element, a torque is transmitted, in addition to pressure and tension, for both a screwing-in and unscrewing movement.

In the primary exemplary embodiment, a two-stage torque monitoring device is integrated directly into the twisting instrument. For the first stage, the neck is designed to be slender and elastic in terms of its geometric shape in conjunction with an appropriate choice of material. Thus, in the final phase of the screwing movement, the user perceives a torsion of more than 10 angular degrees via the actuating element, which twists the neck elastically, but does not cause any further screwing-in movement of the screw or bolt. For this function, the neck is designed as a torsion bar. For this purpose, it can be a smooth cylindrical or alternatively frustoconical rod. If necessary, the neck can also be at least partially a rotation body, the contour of which is also arc-shaped, at least in sections. All transitions of individual neck regions are rounded out to minimize any notch stresses that may arise.

The second stage of torque monitoring forms a predetermined breaking zone. It represents the weakest point between the working part and the drive shaft. If the torque provided for the screw connection is exceeded, the neck separates from the drive shaft at the predetermined breaking point. The breaking point is located far outside the already mounted prosthetic denture, such that the neck, together with the working part, can be pulled out of the implant post, for example, with the help of tweezers—without exerting force.

Further details of the invention will be apparent from the subclaims and the following description of schematically illustrated embodiments.

### BRIEF DESCRIPTION OF THE DRAWINGS

FIG. 1: Perspective view of a twisting instrument;

FIG. 2: Enlarged view of the working part according to FIG. 1;

FIG. 3: Longitudinal section of the semi-finished working part after turning, magnified 20 times;

FIG. 4: Longitudinal section of the working part after the groove has been created to produce the tooth gaps, magnified 20 times;

FIG. 5: Cross-section of the working part at the point of the largest diameter, magnified 20 times;

FIG. 6: Partial view of the instrument neck with a partial section, magnified 30 times;

FIG. 7: Side view of a forming tool, magnified 20 times;

FIG. 8: Engagement of the working part in the screw-in component in section through the external hexagonal teeth, magnified 20 times;

FIG. 9: Engagement of the working part in the screw-in component in section through the external hexagonal tooth gaps, magnified 20 times;

FIG. 10: As in FIG. 8, but the center line of the working part encloses a deflection angle of 22 angular degrees with the center line of the screw-in part;

FIG. 11: As in FIG. 9, but the center line of the working part encloses a deflection angle of 22 angular degrees with the center line of the screw-in part;

FIG. 12: Perspective view of a twisting instrument with a predetermined breaking zone in the form of an annular groove in the drive shaft;

FIG. 13: Longitudinal section to FIG. 12

FIG. 14: Perspective view of a twisting instrument with a predetermined breaking zone in the form of a groove;

FIG. 15: Longitudinal section to FIG. 14;

FIG. 16: Perspective view of a twisting instrument with a predetermined breaking zone in the form of a notch;

FIG. 17: Longitudinal section to FIG. 16;

FIG. 18: Perspective view of a twisting instrument with a predetermined breaking zone in the form of a cone;

FIG. 19: Longitudinal section to FIG. 18;

FIG. 20: Perspective view of a twisting instrument with a predetermined breaking zone in the form of a circumferential groove with a circular sectional cross-section;

FIG. 21: Longitudinal section to FIG. 20;

FIG. 22: Perspective view of a twisting instrument with a predetermined breaking zone in the form of a Mattheck double contour;

FIG. 23: Longitudinal section to FIG. 22;

FIG. 24: Perspective view as in FIG. 22, but with a protective sleeve surrounding the Mattheck double contour;

FIG. 25: Longitudinal section to FIG. 24;

FIG. 26: Perspective view as in FIG. 20, but with a handle;

FIG. 27: Longitudinal section to FIG. 26.

### DETAILED DESCRIPTION

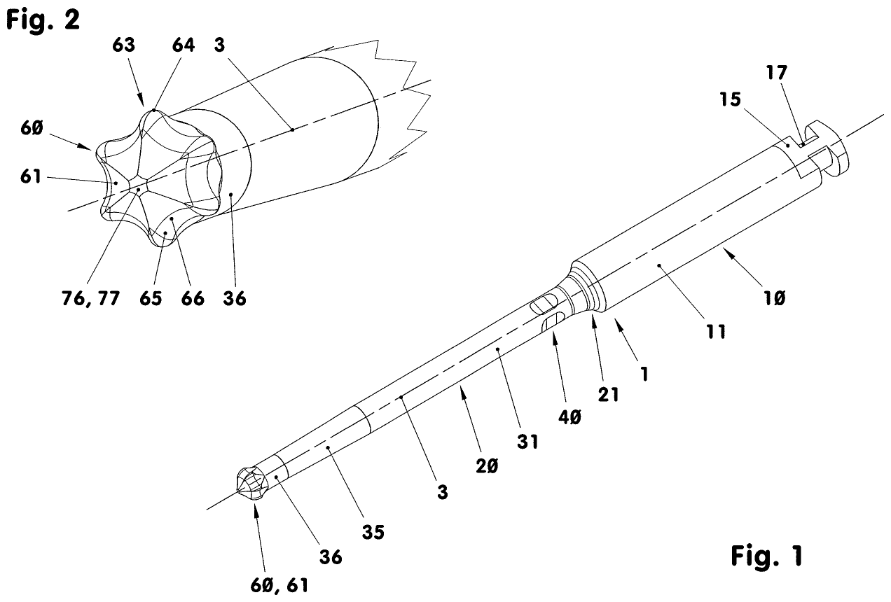

FIG. 1 shows a dental twisting instrument (1) consisting of a drive shaft (10) that is standardized at least in some regions, a slender instrument neck (20) and an external hexagonal spherical head (61) as the working part (60). The twisting instrument (1) can be used to screw a prosthesis to an implant body within the framework of dental implantology for the fabrication of a prosthetic denture.

The one-piece twisting instrument (1) is made, for example, of a cobalt-based austenitic alloy with the material designation COCr20Ni15Mo7. This biocompatible non-magnetic material has a high yield strength and is resistant to corrosion and hydrogen embrittlement. It is resistant to aging and has great fatigue strength.

The twisting instrument (1) is hardened at 530° C. for, for example, three hours in argon or high vacuum and polished afterwards. Depending on the length of the hardening process, the material-related shear modulus is between 75 and 82 GPa.

An alternative material is the high-speed steel HS2-9-1-8. Its main alloying elements are on average, in addition to 1% carbon, 2% tungsten, 9% molybdenum, 1% vanadium and 8% cobalt.

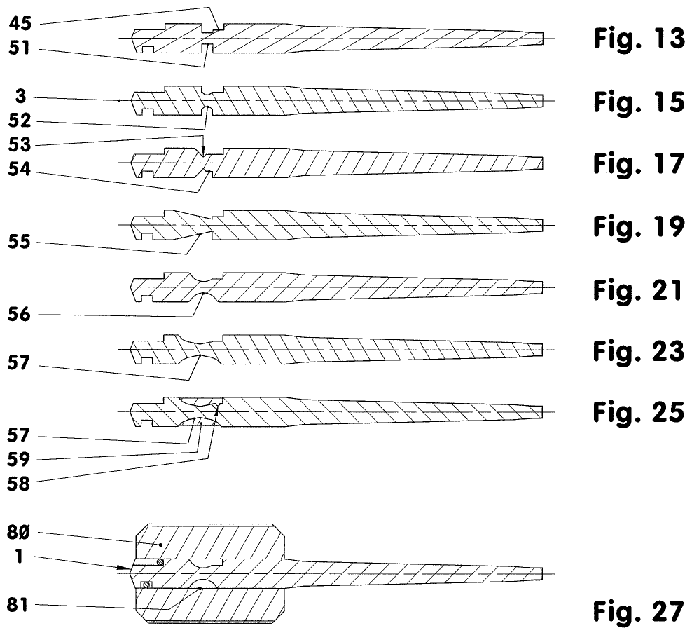

The drive shaft (10) of the twisting instrument (1) is a tool section designed for a rotating or oscillating movement. It has a straight cylindrical shaft section (11), which has a flattening (41) and a groove (17) at its free end. The diameter of the shaft section measures 2.35 mm. In the exemplary embodiment, it has a length of 12.5 mm. A handpiece (80) in the form of an interchangeable handle is placed on the shaft section (11) for use of the twisting instrument (1), see FIGS. 26 and 27. Thereby, the flattening (41) is used to transmit the torque manually introduced into the handpiece (80) to the twisting instrument (1). A pin spring-mounted in the handpiece (80) engages in the groove (17) of the shaft section (11), which serves as a rear grip and holds the twisting instrument (1) in the handpiece (80) such that it cannot be lost. This shaft section (11) is described as type 1 in DIN EN ISO 1779.

Of course, a ratchet mechanism can also be integrated in the handpiece (80). This mechanism makes it possible, for example in a limited working space, to initiate a rotary movement in a specific direction of rotation in the screw-in component (90) by repeated, sector-by-sector swiveling movements of the handpiece (80). In addition, the ratchet mechanism can be combined with a torque limiter.

The shaft section (11) is followed by an instrument neck, for example one that is 16.4 mm long, which is referred to as neck (20) in the following. The neck (20) consists of a neck transition zone (21), a main neck cylinder zone (31), a neck cone zone (35), and a secondary neck cylinder zone (36). In the neck transition zone (21), the shaft section (11) is tapered towards the main neck cylinder zone (31). A diameter reduction from, for example, 2.35 mm to, for example, 1.3 mm takes place. To keep the notch stress caused by the diameter step as small as possible, the neck transition zone (21) is designed using Mattheck's tension triangle method, see FIG. 6.

FIG. 6 shows the neck transition zone (21) located between the shaft section (11) and the predetermined breaking zone (40). In the upper, uncut half of FIG. 6, it can be seen that the neck transition zone (21) consists of the first (23), second (24), and third transition zone regions (25). Thereby, the first transition zone region (23), which adjoins the shaft section (11), has a tapering straight cone with a taper angle of 90 angular degrees. The second transition zone region (24) adjoining the first transition zone region (23) has a tapering straight further cone having a taper angle of 45 angular degrees. The third transition zone region (25), which adjoins the second transition zone region (24), also has a straight tapered cone with a taper angle of 22.5 angular degrees. A transition radius of, for example, 0.5 mm is provided between each taper.

The length of the individual transition zone region (23-25) arises from the design by means of the three dashed tension triangles (26-28)—shaded differently in the sectional view according to FIG. 6. All three tension triangles form an isosceles triangle. The first tension triangle (26), it is right-angled, has a hypotenuse extending like the contour of a 45° chamfer from the edge of the shaft section (11) to the level of the outer diameter of the main neck cylinder zone (31). The acute-angled second tension triangle (27) is connected to the half of the hypotenuse of the first tension triangle (26) that is close to the center line with its cathetus closest to the shaft section (11). The angle enclosed between the hypotenuse and a cathetus measures 22.5 angular degrees. The third tension triangle (28) nestles with its cathetus closest to the shaft section (11) on the half of the hypotenuse of the second tension triangle (27) close to the center line. Its other cathetus is located at the level of the outer diameter of the main neck cylinder zone (31). The two half hypotenuses of the tension triangles (26) and (27) together with the hypotenuse of the third tension triangle (28) form the Mattheck contour (29) with low notch stress. Of course, the Mattheck contour (29) can be approximated by a continuous curve.

The cylindrical main neck cylinder zone (31) has a length of, for example, 10.15 mm.

A predetermined breaking zone (40) is located in the rear region of the main neck cylinder zone (31). The center of the predetermined breaking zone (40) is, for example, 2.25 mm in front of the shaft section (11). The predetermined breaking zone (40) has, for example, three to six flattenings (41) distributed equidistantly around the circumference of the main neck cylinder zone (31). According to FIGS. 1 and 6, four flattenings are selected in the exemplary embodiment. Each flattening is formed flat and has the shape of a rectangle. The rectangular edge length, which is aligned parallel to the center line (3), has a length of, for example, 0.5 mm. The rectangular edge length perpendicular to the center line (3) measures 0.75 mm, for example. The flattenings (41) of the predetermined breaking zone (40), which are adjacent to each other along the circumference, do not contact each other, although their centroids lie on a plane that is intersected perpendicularly by the center line (3). The flattenings thus form a square profile with rounded corner regions.

The rectangular edges lying transverse to the center line (3) are each adjoined—in the longitudinal direction of the twisting instrument (1)—by flattening outlets (42), the concavely curved surfaces of which are here partial surfaces of a cylinder, the individual diameter of which measures, for example, 1 mm in each case.

The cross-section of the predetermined breaking zone (40) is at least 18% smaller than the regular cross-section of the main neck cylinder zone (31).

The neck cone zone (35) adjoining the main neck cylinder zone (31) at the front reduces its diameter towards the working part (60) to, for example, 1.1 mm. The neck cone zone (35) encloses a taper angle of 2.86 angular degrees starting from the main neck cylinder zone (31). The neck cone zone (35) has a length of, for example, 4 mm. It is followed by a secondary neck cylinder zone (36), for example 1 mm long, which extends to the working part (60).

FIGS. 12-27 show twisting instruments that have various predetermined breaking zones (40) in the region of the drive shaft (10) inserted in the handpiece (80) according to FIGS. 26 and 27. A second, 0.9 mm-wide drive flattening (45) is arranged in front of the respective predetermined breaking zone (40), the distance of which from the center line (3) corresponds to the center line distance of the first drive flattening (15). The second drive flattening (45) has the task of serving as an auxiliary drive surface for introducing a torque after the predetermined breaking zone (40) has ruptured. The second drive flattening (45) ends at the front—like the first drive flattening (15)—in a, for example, flat end stop surface (46). Both end stop surfaces (16, 46) are spaced, for example, 4.8 mm apart.

In FIGS. 12 and 13, an annular groove (51), for example, 0.725 mm deep and, for example, 0.9 mm wide, is located immediately behind the second drive flattening (45) as a predetermined breaking zone. The annular groove (51) has a rectangular cross-section.

FIGS. 14 and 15 show, instead of the annular groove (51), a groove (52) of equal width, whose groove base contour is a semicircle. The groove (52) is also 0.725 mm deep.

The predetermined breaking zone of FIGS. 16 and 17 consists of a short cylindrical section (54) and a notch (53), whose notch flanks each enclose an angle of 45 angular degrees with the center line (3). The cylinder section (54) located in front of the sharp notch (53) has a length of 0.53 mm for a diameter of 1.35 mm, for example. The minimum diameter of the predetermined breaking zone amounts to 0.9 mm in the region of the 90° notch. This minimum diameter applies to all variants of FIGS. 12-27.

FIGS. 18 and 19 show, between the second (45) and first drive flattenings (15), a notch cone (55), for example 3.21 mm long, whose taper angle amounts to, for example, 24.4 angular degrees. The narrowest point of the notch cone (55) ends in a flat partial ring surface immediately in front of the second drive flattening (45).

In FIGS. 20 and 21, the predetermined breaking zone forms a circumferential groove (56) with a circular sectional cross-section. The radius of the groove contour measures, for example 1.25 mm. Towards the drive shaft (10), there is a transition radius of, for example, 0.25 mm.

Each predetermined breaking zone of FIGS. 22-25 is a circumferential groove with a Mattheck double contour (57). The simple Mattheck contour (29) is described in connection with the neck transition zone (21) of FIG. 6. In the case of FIGS. 22-25, two Mattheck contours (29) are arranged side by side or mirrored in such a manner that the narrowest tension triangles of both contours contact each other at their acute-angled corners.

The predetermined breaking zone of FIGS. 24-25 is surrounded by a protective sleeve (59) that fills the circumferential groove (57). The cylindrical external wall of the protective sleeve (59) has a diameter that is, for example, 0.15 mm smaller than the diameter of the drive shaft (10). In order to hold the protective sleeve (59) securely against loss on the front part of the drive shaft (10), there is a small retaining recess (58) in the second drive flattening (45), for example in the form of a short bore, in which the material of the protective sleeve (59) engages in a positive-locking manner.

If the predetermined breaking zone breaks, the protective sleeve (59) remains attached to the front part of the twisting instrument (1) in this manner, such that there is no risk of injury to the patient if the handpiece (80) slips out.

FIGS. 26 and 27 show a handpiece (80) into which the twisting instrument shown in FIG. 20 is inserted in such a manner that it cannot be lost or slip. In the sectional view of FIG. 27, it can be clearly seen that the predetermined breaking zone is located in the center region of the mounting hole (81) of the handpiece (80). This ensures that there is no risk of injury to the user of the twisting instrument (1) if the predetermined breaking zone breaks.

The working part (60) is an external hexagonal spherical head (61). The latter is suitable for torque-transmitting engagement in a tool recess (92) of a screw-in component (90) with a hexagon socket according to DIN EN ISO 10664. The external hexagonal spherical head (61) has six teeth (63) on the circumference, between which there are six tooth gaps (66), see also FIG. 2. The tooth heads (64) of the teeth (63) have head roundings that merge directly into the tooth root roundings in the region of the tooth gaps (66). The working part (60) transmits the torque to the internal hexagon (92) of the screw-in component (90) via the flanks (65) of its teeth (63).

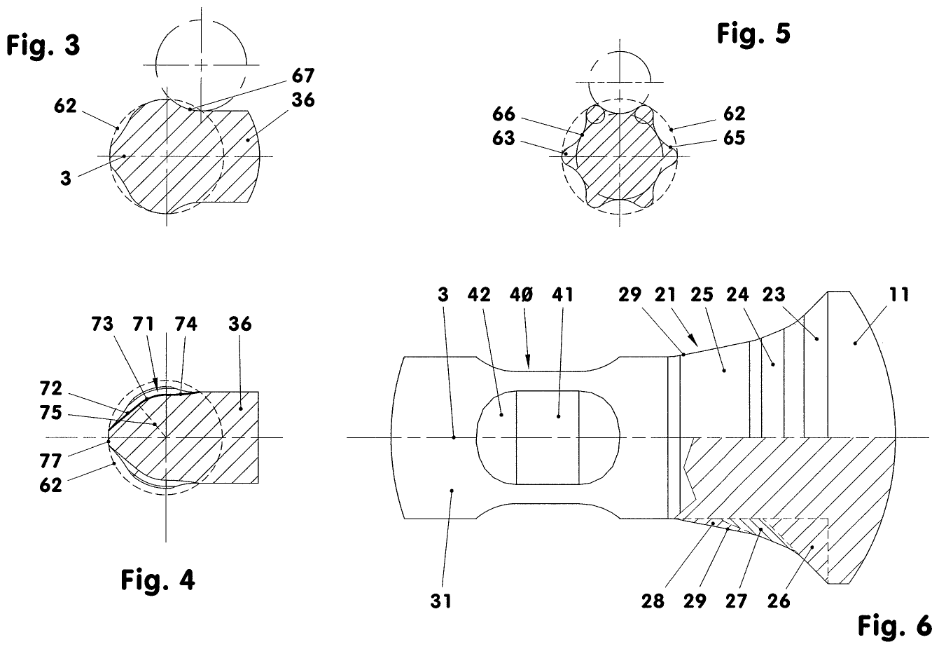

As a semi-finished working part, the working part (60) consists of a ball formed on the front end of the neck (20) or the secondary neck cylinder zone (36)—located on the center line (3), see FIG. 3. In the finished product, the surface of such ball represents the enveloping surface (62) shown as a dashed line in FIGS. 3-5. It has a diameter of, for example, 1.38 mm.

In a first operation, as shown in FIG. 3, a rear grip (67) is created at the transition to the secondary neck cylinder zone (36), which makes it possible to tilt the center line (3) of the twisting instrument (1) by a deflection angle (7) of up to 22 angular degrees relative to the center line (99) of the screw-in component (90). The rear grip (67) has a radius contour whose radius measures 0.55 mm, for example. The radius contour merges tangentially into the secondary neck cylinder zone (36). The center of the radius contour sits, for example, 0.4 mm behind the center point of the enveloping surface (62) lying on the center line (3).

In a further operation, the ball of the semi-finished working part is toothed with the aid of the forming tool (100), see FIG. 4. The forming tool (100) is used to machine the toothing by grinding or milling, depending on the tool type, or to form them in a rolling process.

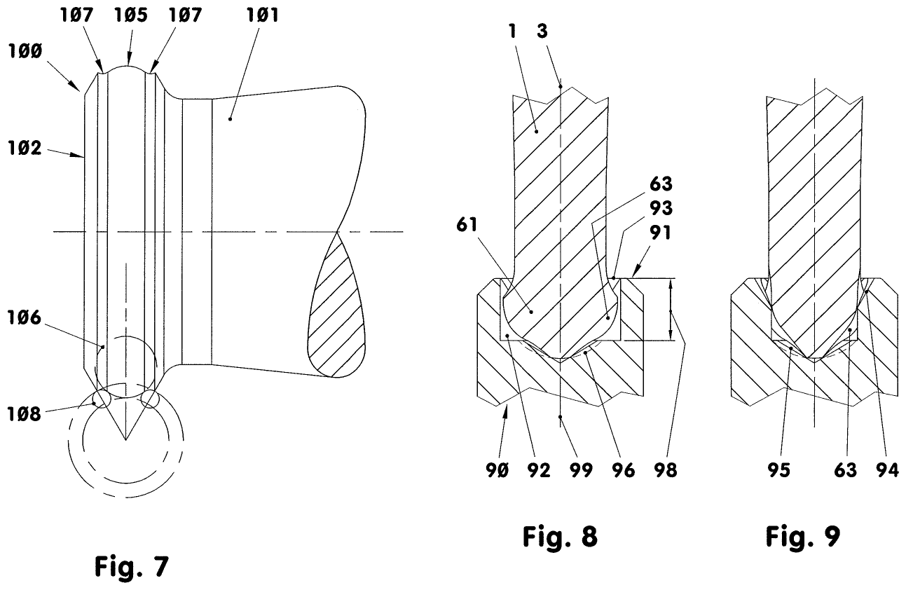

For this purpose, the forming tool (100) has a profile disk (102) formed on a tool shaft (101), see FIG. 7. The profile disk (102), which has a diameter of, for example, 4 mm, has a center tooth gap profile (105) and two tooth head profiles (107) on the edge side. The tooth gap profile (105) is part of a torus surface whose diameter of the gap circle (106) amounts to, for example, 0.74 mm. The tooth head profiles (107) on the edge side are each also part of a toroidal surface whose diameter of the tooth head circles (108) measures, for example, 0.22 mm. The center points of the tooth head circles (108) are spaced apart from the center point of the gap circle (106) by, for example, 0.29 mm—parallel to the center line (3). Secondly, they are arranged in a manner offset radially outwards by 0.384 mm from the center point of the gap circle (106). The tooth head circles (108) each contact the gap circle (106) at a point of contact. In the two points of contact, both circles (106, 108) have identical tangents.

To form the six teeth (63) and the corresponding tooth gaps (66), the forming tool (100) is guided along a base tooth gap contour (71) shown in FIG. 4. The latter consists of a front, straight-line gap base region (72), a center, arc-shaped gap base region (73) and a rear, likewise arc-shaped gap base region (74). All three gap base regions (72-74) merge tangentially.

The center gap base region (73) is a circular arc covering an angle of 40 angular degrees and 26 angular minutes.

The circular arc has a radius of, for example, 0.515 mm. The center point of the center gap base region (73) corresponds to the center point of the spherical enveloping surface (62). The foremost point of the center gap base region (73) lies on a notional radius ray (75) that encloses an angle of 50 angular degrees with the center line (3).

In the foremost point of the center gap base region (73), the straight-line front gap base region (72) joins. The latter ends at the free end (76) of the working part (60). The straight-line gap base region (72) encloses an angle of 40 angular degrees with the center line (3).

The rear gap base region (74) starts at the rear end of the center gap base region (73). It has a radius of, for example, 2.175 mm. The rear gap base region (74) runs out in the rear grip (67) of the working part (60).

After each traverse of the base tooth gap contour (71), the semi-finished working part is swiveled 60 degrees for the next forming process. In this manner, a toothing arises as shown in FIG. 5 as a cross-section through the center point of the enveloping surface (62) and in FIG. 2 in a perspective view. According to FIG. 2, the tip (76) of the working part (60) is a head star (77) whose smallest diameter measures, for example, 0.18 mm. The curvature of the head star (77) corresponds to the curvature of the enveloping surface(62).

All dimensions refer to a hexagon socket for screws according to DIN EN ISO 10884, type no. 5.

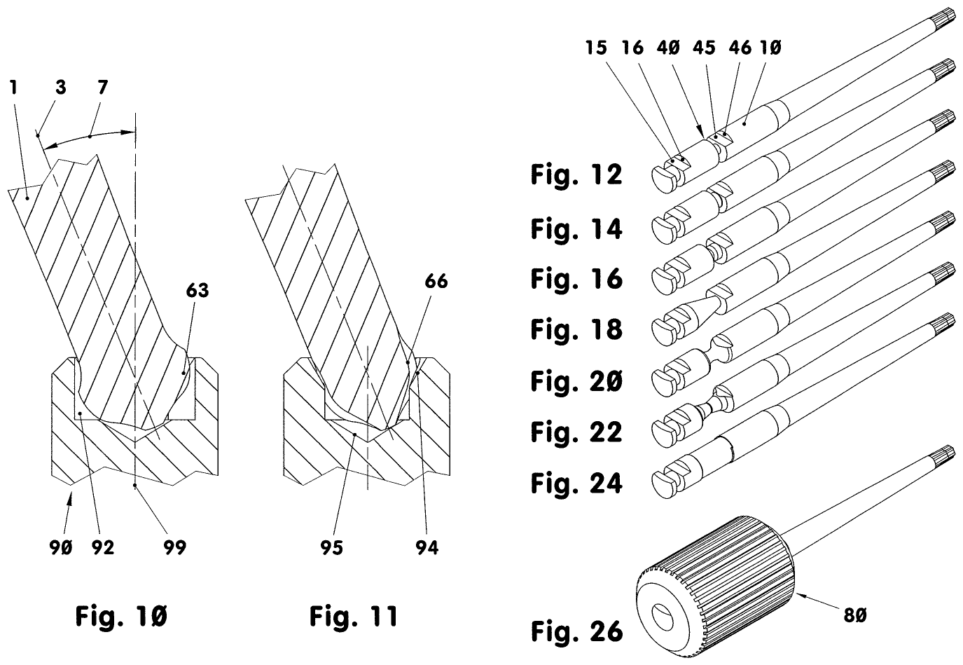

The screw-in component (90) is a screw, threaded bolt or the like, depending on the abutment type. The head of the screw or at least one of the ends of the threaded bolt has a tool recess (92), which is a hexagon socket according to DIN EN ISO 10884, see FIGS. 8-11. According to these figures, the tool recess (92) has a 30° chamfer (94) in the region of its opening (93), the width of which is smaller than half the depth (98) of the useful region of the tool recess (92). The width of the 30° chamfer (94) amounts to, for example, 0.36 mm according to FIGS. 9 and 11.

The tool recess (92) has a base (95) that is in the shape of a straight cone, whose taper angle measures, for example, 118 angular degrees and whose tip (76) is further from the opening (93) of the tool recess (92) than the base cone line. The twisting instrument (1) is supported on this base (95) upon each screwing-in or unscrewing process of the screw-in component (90), in order to ensure that the working part (60) rests securely in the tool recess (92).

Alternatively, a base (96) can be provided in the tool recess (92), the base surface of which is spherically curved; see dashed arc in FIGS. 9 and 10. The curvature of the base (96) has a radius of, for example, 0.7 mm. The center point of the curvature lies, for example, 0.265 mm below the flat end face of the screw-in component (90) surrounding the tool recess (92). If necessary, the base can also be a differently curved rotation surface or even a lowered plane surface.

For screwing in the screw-in component (90)—into the implant body not shown in the figures—the twisting instrument (1) is inserted with its working part (60) into the tool recess (92) of the screw-in component (90). Thereby, on the one hand, the teeth (63) of the working part (60) engage in the tooth gaps of the tool recess (92) with minimal play. On the other hand, the tip (76) or the head star (77) of the working part (60) rests against the base (96) of the screw-in component (90), see FIGS. 8-11.

In FIGS. 8 and 10, the working part (60) of the twisting instrument (1) is cut in the region of the tooth heads (64) of the external hexagonal teeth (63), while in FIGS. 9 and 11 the screw-in component (90) is shown pivoted by ideally, for example, 30 angular degrees about its center line (99). Accordingly, the twisting instrument (1) has also ideally been pivoted by, for example, 30 angular degrees about its center line (3).

The increasing gimbal error with increasing deflection angle (7) causes, at least theoretically, an offset and angular change of the center line (99) of the twisting instrument (1) between FIGS. 8 and 9 and FIGS. 10 and 11. This is neglected here.

Towards the end of the screwing-in process, the tightening torque increases abruptly. In order to make it possible for the user to haptically experience reaching the maximum tightening torque on the handpiece (80) of the twisting instrument (1), the neck (20) of the twisting instrument (1) twists by 10-12 angular degrees according to FIG. 1 without a measurable plastic deformation occurring in the predetermined breaking zone (40).

### LIST OF REFERENCE SIGNS

1 Twisting instrument, screwdriver instrument
3 Center line
7 Deflection angle, angle between (3) and (99)
10 Drive shaft
11 Shaft section, cylindrical
15 Drive flattening, first
16 End stop surface
17 Groove, slot
20 Neck, instrument neck
21 Neck transition zone
23 First transition zone region
24 Second transition zone region
25 Third transition zone region
26 First tension triangle, right-angled
27 Second tension triangle
28 Third tension triangle
29 Mattheck contour
31 Neck cylinder zone, main neck cylinder zone
35 Neck cone zone, neck section
36 Neck cylinder zone, secondary neck cylinder zone, neck section
40 Predetermined breaking zone
41 Flattening, four, flat
42 Flattening outlets
45 Drive flattening, second
46 End stop surface
51 Ring groove
52 Groove
53 Notch, 90° notch
54 Cylinder section
55 Notch cone, cone
56 Circulation groove
57 Mattheck double contour; groove, circumferential
58 Retaining recess
59 Protective sleeve
60 Working part
61 Spherical head, external hexagonal spherical head
62 Enveloping surface, spherical, ball
63 Teeth, external hexagonal teeth
64 Tooth heads
65 Flanks, tooth flanks
66 Tooth gaps
67 Rear grip
71 Base tooth gap contour
72 Front gap base region, straight-line
73 Center gap base region, arc-shaped
74 Rear gap base region, arc-shaped
75 Radius ray of (60), notional
76 Free end, tip
77 Head star
80 Handpiece, handle, actuating element
81 Mounting hole
90 Screw-in component, screw
91 Head, hexagon socket head
92 Tool recess, hexagon socket, Torx®
93 Opening
94 Chamfer, 30° chamfer
95 Base of (92), cone-jacket-shaped
96 Base, spherically curved
98 Depth, penetration depth of (92)
99 Center line of (90)
100 Forming tool
101 Tool shaft
102 Profile disk
105 Tooth gap profile, center
106 Gap circle, circle
107 Tooth head profile, edge side
108 Tooth head circles, circle

1-11. (canceled)

12. A twisting instrument for a screw-in component (90) having a tool recess (92), comprising:
a drive shaft (10);
a neck (20); and
a working part (60),
wherein the working part (60) is a spherical head (61) having a plurality of teeth (63),
wherein a spherical enveloping surface (62) of the working part (60) is contacted by a free end (76) and by sections of tooth heads (64) of the teeth of the working part (60), and
wherein the neck (20) or the drive shaft (10) has a predetermined breaking zone (40).

13. The twisting instrument according to claim 12,
wherein the drive shaft (10) is of type 1 according to DIN EN ISO 1797,
wherein the drive shaft (10) includes a drive flattening (15) and a groove (17) at a further free end.

14. The twisting instrument according to claim 12,
wherein the neck (20) comprises
a neck transition zone (21),
a neck cylinder zone (31, 36), and
a neck cone zone (35).

15. The twisting instrument according to claim 14,
wherein the neck transition zone (21) comprises
a first (23) transition zone region,
a second transition zone region (24), and
a third transition zone region (25),
wherein the first transition zone region (23) is a tapering straight cone with a taper angle of 90 angular degrees adjoining the drive shaft (10),
wherein the second transition zone region (24) is a tapering straight cone having a taper angle of 45 angular degrees adjoining the first transition zone region (23), and
wherein the third transition zone region (25) is a tapered straight cone having a taper angle of 22.5 angular degrees adjoining the second transition zone region (24).

16. The twisting instrument according to claim 12,
wherein a neck cylinder zone (31) adjoining the drive shaft (10) has a length corresponding to at least 6.6 times a diameter of the neck cylinder zone (31) and
wherein a neck section located between the neck cylinder zone (31) and the working part (60) has a length that is at least 5.5 times a smallest diameter of the neck section.

17. The twisting instrument according to claim 14,
wherein the predetermined breaking zone (40) is arranged in a rear region of the neck cylinder zone (31) adjoining the neck transition zone (21).

18. The twisting instrument according to claim 14,
wherein the predetermined breaking zone (40) has four flattenings (41) distributed equidistantly around a circumference of the neck cylinder zone (31) adjoining the neck transition zone (21).

19. The twisting instrument according to claim 18,
wherein adjacent one of the flattenings (41) of the predetermined breaking zone (40) do not contact each other.

20. The twisting instrument according to claim 12,
wherein the spherical head (61) is an external hexagonal spherical head.

21. A system, comprising
the twisting instrument according to claim 12; and
a screw-in component with a tool recess,
wherein the tool recess (92) has in a region of an opening (93) a 30° chamfer (94), a width of which is smaller than half of a depth (98) of a useful region of the tool recess (92).

22. The system as in claim 21,
wherein the tool recess (92) has a base that is in the shape of a straight cone, whose taper angle measures 116-120 angular degrees and whose tip is further from the opening (93) than the base cone line.
